(19) United States
(12) Patent Application Publication
(10) Pub. No.: US 2017/0253914 A1
(43) Pub. Date: Sep. 7, 2017

(54) APPARATUS, SYSTEM, AND METHOD FOR DISPENSING OR MIXING MICRO QUANTITY OF LIQUID

(71) Applicant: INSTITUTE OF MICROBIOLOGY, CHINESE ACADEMY OF SCIENCES, BEIJING (CN)

(72) Inventors: WENBIN DU, BEIJING (CN); JUANLI YUN, BEIJING (CN); QIAOLIAN YI, BEIJING (CN); PENG XU, BEIJING (CN); XIAOWEI ZHENG, BEIJING (CN); XIN DAI, BEIJING (CN); LI HUANG, BEIJING (CN)

(73) Assignee: INSTITUTE OF MICROBIOLOGY, CHINESE ACADEMY OF SCIENCES, BEIJING (CN)

(21) Appl. No.: 15/598,186

(22) Filed: May 17, 2017

Related U.S. Application Data
(63) Continuation-in-part of application No. PCT/CN2015/077630, filed on Apr. 28, 2015.

(30) Foreign Application Priority Data
Nov. 17, 2014 (CN) 201410655191.5
Nov. 17, 2014 (CN) 201410655309.4
Dec. 27, 2016 (CN) 201611226046.0
Dec. 27, 2016 (CN) 201611227062.1
Dec. 27, 2016 (CN) 201611227882.0
Dec. 27, 2016 (CN) 201611227913.2

(51) Int. Cl. C12Q 1/68; B01L 3/02; G01N 35/00; G01N 35/10

(52) U.S. Cl. CPC C12Q 1/6806; G01N 35/10; B01L 3/021; G01N 35/00584; B01L 2300/0838; B01L 2200/143

(57) ABSTRACT

An apparatus for dispensing or mixing micro-quantity of liquid includes a fluid driving unit having a fluid driving device, and a mechanical moving unit. The fluid driving device and the mechanical moving unit cooperate with each other to have a micro-quantity of the first liquid driven out from and attached to an outlet end of the micro-pipe in the second liquid, and to have the micro-quantity of the first liquid detached from the outlet end by drawing the outlet end of the micro-pipe out from the second liquid. A system and method for dispensing or mixing micro-quantity of liquid is also disclosed.

### CROSS-REFERENCE TO RELATED APPLICATIONS

This application claims all benefits accruing under 35 U.S.C. §119 from China Patent Applications No. 201410655309.4, filed on Nov. 17, 2014, No. 201410655191.5, filed on Nov. 17, 2014, No. 201611226046.0, filed on Dec. 27, 2016, No. 201611227062.1, filed on Dec. 27, 2016, No. 201611227913.2, filed on Dec. 27, 2016, and No. 201611227882.0, filed on Dec. 27, 2016 in the State Intellectual Property Office of China, the contents of which are hereby incorporated by reference. This application is a continuation in part under 35 U.S.C. §120 of international patent application PCT/CN2015/077630 filed on Apr. 28, 2015, the content of which is also hereby incorporated by reference.

### FIELD

The present disclosure relates to biochemical detection and screening, and more particularly to apparatuses, systems, and methods for dispensing or mixing micro quantities of liquids.

### BACKGROUND

Life sciences involve researches on genetic engineering, protein engineering, and drug screening. When conducting the researches, micro quantities of liquids containing nucleic acids, proteins, and other organic and inorganic substances are transferred and dispensed frequently.

In cell physiology, studies are performed in cellular and molecular levels. Commonly used methods in single-cell analysis involve and include: (1) microsurgical technology; (2) laser capture microdissection; (3) laser induced fluorescence detection based flow cytometry; and (4) microfluidics.

The microsurgery and laser capture microdissection are performed by manually isolating cells or particles with different optical characteristics under a microscope. However, the throughput is low, and operations may destroy the integrity of the sample.

The laser induced fluorescence detection based flow cytometry is a high-throughput single cell sorting method with a general analysis speed of 5,000 to 10,000 cells per second. Cells that pass through the flow cytometer are dispersed into nanoliter to picoliter droplets, and can be assigned to the microplate one by one for subsequent reactions and analysis.

The field of microfluidics has developed rapidly in recent years. Droplets are generated in a microfluidic channel based on interface discontinuity between the dispersed phase and the continuous phase. Droplets with uniform size can be generated, integrated, reacted, and sorted through different microfluidic channel designs. The design, processing, and operation of the microfluidic chip relates to factors such as specific flow rate, oil-water interface tension, channel configuration, channel surface modification, and so on.

### SUMMARY

According to one aspect of the present disclosure, a micro-quantity liquid dispensing/mixing apparatus is provided. The micro-quantity liquid dispensing/mixing apparatus comprises a fluid driving unit and a mechanical moving unit.

The fluid driving unit comprises a fluid driving device configured to drive a first liquid in a micro-pipe to an outlet end of the micro-pipe. The micro-pipe can be connected to the fluid driving device.

The mechanical moving unit is configured to control the micro-pipe and/or the container containing the second liquid to move in at least one dimension to cooperate with a start and a stop of the fluid driving device to have the outlet end of the micro-pipe inserted into and left from the second liquid, thereby dispensing the predetermined volume of the first liquid in the container.

The first liquid is not soluble with the second liquid.

In one embodiment, the fluid driving unit can further comprise a passage member. The passage member can be joined between the fluid driving device and at least one micro-pipe. The passage member can define one or more fluid passages. In one embodiment, the passage member defines an array of fluid passages arranged corresponding to the containers. One passage member can be connected to one or more fluid driving devices. Each fluid passage is connected to and in fluid communication with one micro-pipe. In one embodiment, the passage member comprises an array of fluid passages arranged according to a standard microplate (e.g., 24, 96, 384, or 1536 well) such that the liquid can be dispensed to and/or mixed in the standard microplate, which is compatible to conventional equipment and apparatuses for further reaction/examination.

The fluid driving unit having the passage member with a plurality of fluid passages is configured to having the first liquid driven to a plurality of micro-pipes at the same time, thereby dispensing the micro-quantities of first liquid in a plurality of containers at the same time to achieve a high-throughput dispensing of the first liquid.

In one embodiment, the fluid driving unit can comprise two or more fluid driving devices, and each fluid driving device is connected to at least one micro-pipe, such that different kinds of liquids and/or different volumes of liquids can be dispensed at the same time to improve the dispensing efficiency.

In one embodiment, one fluid driving device can drive the first liquid in each or a plurality of fluid passages (e.g., a row or a column of fluid passages) in the fluid passage array.

The mechanical moving unit can comprise a manipulator for controlling the movement of the micro-pipe and/or a moving support for controlling the movement of the container. The manipulator and the moving support can be respectively movable in at least one dimension to move the micro-pipe and/or the container therewith. The mechanical moving unit can have a stable relative motion between the micro-pipe and the container, such as in a uniform speed, to avoid a shock to the system and affecting the dispensing accuracy.

The micro-quantity liquid dispensing/mixing apparatus can further comprise a control and feedback unit. The control and feedback unit can comprise a signal receiving module, a calculating module, and a signal outputting module.

The signal receiving module is configured to receive a signal and transmit the signal to the calculating module. The calculating module is configured to calculate the signal and/or compare the signal with a set value, and then transmit a calculation result and/or a comparison result to the signal outputting module. The signal outputting module is configured to receive the calculation result and/or comparison result, convert the result into an instruction signal, and output the instruction signal.

The signal can be a volume signal representing a volume of the liquid that is to be dispensed into, such as a volume of the droplet to be generated. The calculating module can decide a suitable fluid driving speed based on the volume signal, or the fluid driving speed is predetermined. The calculating module can further calculate the operating time of the fluid driving device corresponding to the fluid driving speed and the volume of the liquid to be dispensed. The signal outputting module receives the calculated operating time from the calculating module and outputs the corresponding instruction signals respectively to the fluid driving unit and the mechanical moving unit to control the cooperation between the fluid driving unit and the mechanical moving unit to complete the dispensing of the first liquid.

In one embodiment, the control and feedback unit can further comprise a distance detecting module configured to obtain a relative positional relationship signal and/or a distance signal. The relative positional relationship signal represents a relative positional relationship between the outlet end of the micro-pipe and a target container. The distance signal represents a distance between the outlet end of the micro-pipe and a liquid surface of the second liquid in the target container. The control and feedback unit can transmit the relative positional relationship signal and/or the distance signal to the signal receiving module. The signal receiving module can transmit the relative positional relationship signal and/or the distance signal to the calculating module. The calculating module can compare the relative positional relationship signal and/or the distance signal with a preset distance between the outlet end of the micro-pipe and the liquid surface of the second liquid in the target container. The signal outputting module can receive the compared result from the calculating module, convert the compared result into corresponding instruction signals, and transmit the instruction signals to the mechanical moving unit to adjust the movement of the mechanical moving unit until the outlet end of the micro-pipe is located at a predetermined distance above the liquid surface of the second liquid.

In one embodiment, the distance detecting module is capable of obtaining a depth signal representing the depth of the second liquid in the container and transmitting the depth signal to the signal receiving module. The signal receiving module can transmit the depth signal to the calculating module. The calculating module can compare the depth of the second liquid with a preset minimal depth. When the depth of the second liquid is larger than the preset minimal depth, the signal outputting module transmits the instruction signals to the mechanical moving unit and the fluid driving unit to start to dispense the first liquid. When the depth of the second liquid is smaller than the preset minimal depth, the signal outputting module transmits the instruction signals to the mechanical moving unit and the fluid driving unit to stop dispensing the first liquid, and can also output an alarm signal in some embodiments. In one embodiment, the fluid driving unit can dispense the second liquid instead of the first liquid when the depth of the second liquid is smaller than the preset minimal depth.

The control and feedback unit is configured to precisely control the operation of the mechanical moving unit and the fluid driving unit to improve the accuracy and precision of dispensing the liquid. In the embodiment of the apparatus having a plurality of fluid driving devices, especially when the fluid driving unit comprises a plurality of fluid driving devices, and different fluid driving devices are configured to dispense different volumes of liquid, the control and feedback unit can automatically complete complex and varied liquid dispensing schedules.

The micro-quantity liquid dispensing/mixing apparatus can further comprise a sterilization/dedusting/destaticizing and gas replacing unit. The sterilization/dedusting/destaticizing and gas replacing unit can comprise a filtering device that is capable of forming a closed space enclosing the micro-pipe and the container. The sterilization/dedusting/destaticizing and gas replacing unit can also comprise at least one of an ultraviolet sterilizing light and an air filter. The ultraviolet sterilizing light surrounds the micro-pipe and the container. The sterilization/dedusting/destaticizing and gas replacing unit can further comprise a gas replacing device configured to replace the gas in the apparatus, thereby having inert gas protection and anaerobic operation, and providing a gas atmosphere required for cell culture or biochemical reaction such as carbon dioxide, oxygen gas, or hydrogen gas.

In one embodiment, the micro-quantity liquid dispensing/mixing apparatus can further comprise a temperature control and feedback unit configured to heat and/or cool the container to provide a desired temperature for the liquid dispensing. The temperature control and feedback unit can comprise at least one of heating device, cooling device, and temperature controlling device. In one embodiment, the temperature control and feedback unit is an incubator.

In one embodiment, the micro-quantity liquid dispensing/mixing apparatus consists of or consists essentially of the fluid driving unit, the mechanical moving unit and the control and feedback unit.

The micro-quantity liquid dispensing/mixing apparatus has a simplified structure and operation. A conventional micro-quantity liquid dispensing/mixing apparatus often comprises a device for detaching a given volume of droplet from the micro-pipe, such as a piezoelectric device, a heating device, an injection device, and the like. In the present micro-quantity liquid dispensing/mixing apparatus, the fluid driving unit generates a droplet of the first liquid attached to the outlet end of the micro-pipe in the second liquid, and the mechanical moving unit draws the outlet end of the micro-pipe out from the second liquid through the liquid surface, causing the generated droplet detached from the outlet end of the micro-pipe and thereby being dispensed into the second liquid.

The micro-quantity liquid dispensing/mixing apparatus in the present disclosure has a simple structure and is easy to operate. The amount of the dispensed liquid can be adjusted in a wide range from a femtoliter level to a nanoliter level. Moreover, the amount of the dispensed liquid is accurate, and can meet the needs of testing and experiment.

In the embodiment that has the passage member with the plurality of fluid passages, the plurality of fluid passages can be an array corresponding to a standard microplate. The first liquid can be dispensed in all the wells of the microplate at the same time to achieve a high-throughput dispensing of the first liquid, and the dispensing is compatible to conventional equipment and apparatuses for further reaction/detection.

According to another aspect of the present disclosure, a micro-quantity liquid dispensing system is provided. The micro-liquid dispensing system comprises the above-described micro-quantity liquid dispensing/mixing apparatus, a micro-pipe, and a container. The micro-pipe is connected to the fluid driving unit for dispensing the first liquid contained therein. The container contains the second liquid for receiving the dispensed droplet of the first liquid.

In one embodiment, the micro-pipe defines a cylindrical opening or a tapered opening at the outlet end.

The micro-pipe can be a single-core capillary tube, a multi-core (or multi-cell) capillary tube, a capillary tube bundle, a capillary tube array, a sleeve tube enclosed capillary tube, or a microfluidic chip with a tapered probe structure. In one embodiment, the micro-pipe has an enlarged upper end as a reservoir. A material of the capillary tube can be such as glass or quartz. A plurality of capillary tubes can be stacked or bundled together forming the capillary tube bundle or array.

The outlet end with the opening of the micro-pipe can be treated to have a low surface energy. In one embodiment, the outlet end with the opening of the micro-pipe can be silanized to have the droplet detached from the micro-pipe more smoothly.

The container in the micro-quantity liquid dispensing system can be any suitable container. The container can be in form of a single container, a one-dimensional array of containers, or two-dimensional array of containers. In one embodiment, a standard microplate with 24, 96, 384, or 1536 wells can be used as the container array. Corresponding to the container array, a plurality of micro-pipes can be arranged in a one-dimensional array or a two-dimensional array, which improve the throughput for dispensing the first liquid.

In one embodiment, the container has a pointed bottom, a tapered bottom, a rounded bottom, or a flat bottom. The pointed bottom, the rounded bottom, and the similar shaped bottom facilitate locating the droplet to the center of the bottom, not only conducive to fusion and reaction between droplets, but also conducive for a testing equipment to quickly and accurately capture the droplet.

The specific gravity of the first liquid can be greater than the specific gravity of the second liquid. The density of the first liquid can be greater than the density of the second liquid. The droplets of the first liquid can sink to the bottom of the container.

According to yet another aspect of the present disclosure, a method for dispensing micro-quantity of liquid is disclosed, and the method comprises: A, inserting the outlet end of the micro-pipe into the second liquid contained in the container, the first liquid is filled at least in the outlet end of the micro-pipe;

B, driving the first liquid in the micro-pipe to flow towards the outlet end, and forming a droplet of the first liquid having a predetermined volume outside the outlet end of the micro-pipe; and C, drawing the outlet end of the micro-pipe out from the second liquid to detach the droplet having the predetermined volume from the outlet end of the micro-pipe thereby dispensing the droplet in the second liquid;

wherein the first liquid is not soluble with the second liquid.

The droplet of the first liquid is detached from the outlet end of the micro-pipe and dispensed in the second liquid under the action of the fluid shearing force of the second liquid and the surface tension at the interface between the second liquid and a gas phase, or the interface between the second liquid and the third liquid, which is immiscible and covers above the second liquid. The gas phase can be air in the environment that the container is disposed.

A volume of the droplet of the first liquid can be in a range from about 10 femtoliters (fL) to about 10 microliters (μL), and in some embodiments in a range from about 2 picoliters (pL) to about 200 nanoliters (nL).

In some embodiments, the container is the two-dimensional container array, and the micro-pipe is a two-dimensional micro-pipe array correspondingly. In some embodiments, the container is a well on the standard microplate, such as the microplate with 24, 96, 384, or 1536 wells. In some embodiments, the container has a pointed bottom, a tapered bottom, a rounded bottom, or a flat bottom.

In some embodiments, the specific gravity of the first liquid is greater than the specific gravity of the second liquid.

In one embodiment, the container further contains a third liquid. The third liquid can be covered above the second liquid, and is not soluble with both the first and second liquids. In another embodiment, the third liquid can be located below the second liquid, and is not soluble with the second liquid.

In the present disclosure, the micro-quantity of liquid attached outside the outlet end of the micro-pipe is detached from the micro-pipe due to the interface energy of the gas-liquid interface or the liquid-liquid interface, enabling the micro-quantity of liquid to overcome the surface tension of the micro-quantity of liquid at the outlet end of the micro-pipe. A size of the droplet is controlled by the fluid driving unit, and the precision is affected by the size of the opening of the outlet end and the precision of the fluid driving device. By simply inserting and drawing the outlet end of the micro-pipe in and out the second liquid which is not soluble with the first liquid, the first liquid can be dispensed into the droplet in femtoliters to nanoliters.

The amount of the liquid to be dispensed into (i.e., the size of the droplet) can be controlled by controlling the flow speed (or flow rate) and the flow time of the liquid in the micro-pipe. Therefore, the dispensing volume of the liquid can be flexibly adjusted as desired. The micro-pipes can be arranged as an array (e.g., by using a passage member comprising a plurality of fluid passages arranged as an array), in which case a standardized container such as a microplate can be used. Thus, a high-throughput screening and a standardized biochemical reaction and detection of the micro-quantity of liquid can be realized.

For example, for single cell lysis, a microdroplet of the lysis buffer can be added and fused with a single cell to prepare nucleic acids, proteins, and other substances of the single cell. The nucleic acids or proteins can be diluted in an enlarged droplet and taken out, or retained in the second liquid for reactions, which can be a DNA analysis, an RNA analysis, a protein analysis, etc. Corresponding droplets can be added according to varied purposes. The droplets can be enclosed in an oil phase and not be evaporated, which is conducive to the storage. Each reaction can be done independently in one well, avoiding cross contamination, and each reaction can be done in nanoliter sized droplet, greatly decreasing the cost.

### BRIEF DESCRIPTION OF THE DRAWINGS

Implementations will now be described, by way of example only, with reference to the attached figures.

FIG. 1 is a schematic structural view of one embodiment of a method for dispensing micro-quantity of liquid.

FIG. 2 is a schematic structural view of one embodiment of a micro-liquid dispensing/mixing apparatus.

FIG. 3 is a schematic structural view of one embodiment of a two-dimensional container array, micro-pipe, and passage member.

FIG. 4 is a schematic structural view of one embodiment of a double-core capillary tube dispensing liquid.

FIG. 5 is a schematic structural view of one embodiment of a capillary tube bundle consisted of three single-core capillary tubes dispensing liquid.

FIG. 6 is a schematic structural view of one embodiment of a sleeve tube enclosed capillary tube dispensing liquid.

FIG. 7 is a schematic structural view of one embodiment of a microfluidic chip having four joined channels dispensing liquid through its tapered probe structure.

FIG. 8 is a schematic structural view of one embodiment of another microfluidic chip capable of forming double-emulsion droplet dispensing liquid.

FIG. 9 is a schematic structural view of one embodiment of a method for dispensing two solutions into a container and fusing the two solutions together.

FIG. 10 shows microscopic image of two 5 nL droplets in different colors fusing into one 10 nL droplet according to the method of FIG. 9.

FIG. 11 is a graph showing a diameter change from the two nL droplets to the one 10 nL droplet.

FIG. 12 is a schematic structural view of one embodiment of a method for dispensing a microdroplet into a relatively large system.

FIG. 13 is a schematic structural view of one embodiment of a method for dispensing two first liquids into a second liquid under a cover of a third liquid.

FIG. 14 is a schematic structural view of one embodiment of a control module of the micro-quantity liquid dispensing/mixing apparatus.

FIG. 15 shows a flow chart of one embodiment of a method for dispensing/mixing micro-quantity of liquid.

FIG. 16 shows schematic side views of embodiments of containers.

FIG. 17 is a schematic structural view of another embodiment of the micro-liquid dispensing/mixing apparatus.

FIG. 18 is a schematic structural view of one embodiment of a method for aspirating the first liquid into a micro-pipe.

FIG. 19 is a schematic structural view of a method for amplifying micro-quantity of nucleic acid of cell.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout this disclosure are presented as follows.

The term “liquid” refers to a substance in liquid state. The liquid may be a solid at room temperature and a liquid at an elevated temperature. The liquid may also be a gas at room temperature and a liquid at a lowered temperature. The liquid is not limited to a liquid state pure substance, but can also be a liquid solution or a liquid mixture.

The term “first liquid” refers to the liquid to be dispensed, and is not limited to one liquid, but can be, for example, varied kinds of liquids, such as liquid solutions separately made from different samples; and can also be a liquid mixture of varied kinds of liquids. The first liquid can be, but not limited to, a water solution or an aqueous solution, and can also be a hydrophobic liquid that is insoluble in the second liquid.

The term “second liquid” refers to the liquid that is used to “cut” the first liquid thereby having the droplet of the first liquid detached from a micro-pipe, and also used to carry the droplet of the first liquid. In some embodiments, the second liquid also provides a required environment for a reaction, such as oxygen isolation, including dark, warm, etc. The second liquid is insoluble and inert with the first liquid, and can be a non-volatile oil.

The term “third liquid” and “other liquid” both refer to the liquid other than the first and second liquids. The third liquid can be a liquid associated with the first liquid, such as a diluent for diluting the first liquid, a bulky reactant for reacting with the first liquid; and can also be a liquid associated with the second liquid, such as a liquid seal for the second liquid. The third liquid can also be a sealing liquid filled in the micro-pipe to seal the first liquid from a side away from an outlet end of the micro-pipe.

The term “micro-quantity”, for example in micro-quantity of liquid, refers to an amount of liquid that is not able to be precisely and repeatedly dispensed by a regular pipette, such as liquid smaller than a microliter level, and in some embodiments from a femtoliter level to a nanoliter level.

The term “micro-pipe” refers to any form of pipe including at least one liquid pathway that is used to dispense the first liquid. The most common and the lowest cost micro-pipe is a capillary. The micro-pipe can also be a chip having one or a plurality of microchannels as liquid pathways. The micro-pipe can also be a tube including at least one micro-pipe and a sleeve tube encasing the micro-pipe therein.

The term “container” refers to any form of container that is used to contain the second liquid and receive the first liquid. The container can be a well on a microplate. In some embodiments, the container has a narrower bottom compared with the top, such as a pointed bottom, a round bottom, or an oval bottom.

The present disclosure provides an apparatus, a system, and a method that are capable of precisely dispensing micro-quantity of liquid. Different from macroscopic volume such as in the milliliter level or the microliter level, when the volume of liquid becomes very small, such as in a nanoliter level, a picoliter level, or even a femtoliter level, the liquid cannot be directly/automatically “dropped” from a pipe under gravity for the reason that the gravity of the micro-quantity of liquid is far less than the adhesion force and the surface tension between the liquid and the pipe, and far less than the surface tension between the liquid inside and outside the pipe.

In the present disclosure, one embodiment of a micro-quantity liquid dispensing/mixing apparatus comprises a fluid driving unit and a mechanical moving unit.

The fluid driving unit comprises a fluid driving device configured to drive a first liquid in the micro-pipe to an outlet end of the micro-pipe. The micro-pipe can be connected to the fluid driving device.

The mechanical moving unit is configured to control the micro-pipe and/or the container containing the second liquid to move in at least one dimension to cooperate with start and stop of the fluid driving device to have the outlet end of the micro-pipe inserted into and left from the second liquid, thereby dispensing the predetermined volume of the first liquid in the container. The first liquid is insoluble with the second liquid.

The micro-pipe comprises an inlet end connected to the fluid driving unit and an outlet end away from the fluid driving unit. The outlet end is a free end. The first liquid can be previously loaded in the micro-pipe, and filled at least in the outlet end or fully filled in the micro-pipe. The first liquid can be driven from the inlet end by the fluid driving device into the micro-pipe or can be aspirated (e.g., sucked, pulling, by suction) from the outlet end into the micro-pipe. The second liquid, which is insoluble and inert to the first liquid, can be previously loaded in the container.

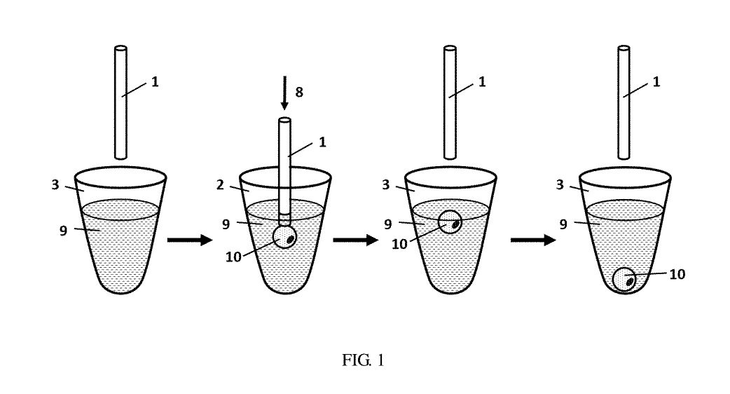

Referring to FIG. 1, the mechanical moving unit is configured to have the micro-pipe 1 and the container 3 having a relative motion therebetween, such that an outlet end of the micro-pipe 1 can be inserted into the second liquid 9. The first liquid 8 can be loaded in the micro-pipe 1. According to the predetermined volume of the first liquid 8 that is to be dispensed into the container 3, a flow speed and a flow time of first liquid 8 can be controlled by the fluid driving unit to drive a micro-quantity of the first liquid 8 with the predetermined volume outside the outlet end of the micro-pipe 1 in the second liquid 9 to form a droplet 10. The droplet 10 is joined to the outlet end of the micro-pipe 1 in the second liquid 9. The droplet 10 attached to the outlet end of the micro-pipe 1 in the second liquid 9 can be spaced from the bottom and the side wall of the container 3. The mechanical moving unit is configured to have the micro-pipe 1 and the container 3 having another relative motion therebetween, such that the outlet end of the micro-pipe 1 is drawn out from the second liquid 9. When the outlet end of the micro-pipe 1 is moving through the liquid surface of the second liquid 9, the droplet 10 is “cut” from the outlet end of the micro-pipe 1 by the interface energy and the shearing force applied to the droplets from the second liquid 9 and the gas phase. The gas phase can be air in the environment. That is, the droplet 10 is detached from the outlet end of the micro-pipe 1 and remains in the second liquid 9 in the container 3. In one embodiment, the specific gravity of the first liquid 8 is larger than the specific gravity of the second liquid 9, such that the droplet 10 of the first liquid sinks to the bottom of the container 3. As the droplet 10 has a small size, the container 3 with the pointed bottom or round bottom is easy for a testing equipment to capture the droplet 10.

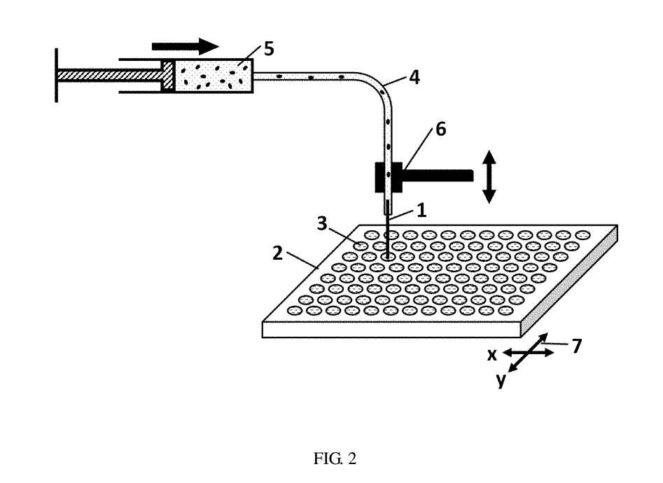

Referring to FIG. 2, in one embodiment, the fluid driving device 5 is a syringe, the passage member 4 is a Teflon® or polytetrafluoroethylene tube connected to and in fluid communication with the output end of the syringe, and the micro-pipe 1 is a capillary tube connected to and in fluid communication with the Teflon® tube. The capillary tube can be vertically arranged and the outlet end is the lower end. The first liquid 8, from which gas bubbles are removed, is fully filled in the syringe, the Teflon® tube, and the capillary tube. The mechanical moving unit can comprise a manipulator 6 and a moving support 7. The manipulator 6 controls the capillary tube to vertically move along a z-axis up and down. The moving support 7 carries a container array 2 comprising a plurality of containers 3 to horizontally move along an x-axis and/or y-axis. The container array 2 can be a microplate comprising wells as the containers 3.

### Fluid Driving Unit

The fluid driving unit comprises the fluid driving device. Besides the syringe shown in FIG. 2, the fluid driving device can also be any device that is capable of continuously driving the first liquid in the micro-pipe, such as a peristaltic pump, a pressure driven pump, a gas driven pump, or an electroosmotic pump. The fluid driving device can have a relatively high fluid driving accuracy to obtain the droplet in the volume of femtoliters to nanoliters.

A fluid driving speed of the fluid driving device can be in a range from about 0.5 picoliter per minutes (pL/min) to about 10 milliliters per minutes (mL/min), and in one embodiment is in a range from about 10 nanoliters per seconds (nL/s) to about 100 nanoliters per minutes (nL/min). In order to obtain a liquid with a high precision of volume, the accuracy error of the liquid flow speed can be ±1% or less, such as less than or equal to ±0.5%, because the droplet to be formed is very small.

The fluid driving unit can further comprise a passage member. The passage member can be joined between the fluid driving device and at least one micro-pipe. The passage member can comprise one or more fluid passages. The passage member 4 can have a single fluid passage such as the Teflon® tube in FIG. 2. In another embodiment, the passage member can have multiple fluid passages respectively communicating with multiple micro-pipes or with the micro-pipe having multiple liquid pathways in order to dispense the liquid to multiple containers simultaneously or dispense multiple kinds of liquids simultaneously. For example, the passage member can comprise an array of fluid passages arranged corresponding to the arrangement of the wells on the microplate.

One end as an inlet end of the passage member can be permanently or removably fixed to the fluid driving device. The connection between the passage member and the fluid driving device can be hermetic to the outside environment.

The other end as an outlet end of the passage member can be capable of having an air-tight connection with the micro-pipe such as a threaded connection, a clamping connection, an interference fit, or a plug connection. A sealant agent can be coated at the joint to insure the air tightness. As the micro-pipe is usually disposable, the outlet end of the passage member can have a structure that is easy to quickly assemble to and remove from the micro-pipe.

A size of the passage member is not limited and can be decided according to the flow speed of the liquid and the volume the liquid to be dispensed into.

In one embodiment, the passage member can be a flexible Teflon® tube having an inner diameter of about 300 microns, an outer diameter of about 600 microns, and a length of about 15 centimeters, and the micro-pipe can be a capillary tube having an outer diameters in a range from about 300 microns to about 400 microns, which can achieve the air-tight connection therebetween simply by inserting the end of the capillary tube into the end of the Teflon® tube without using the sealant agent.

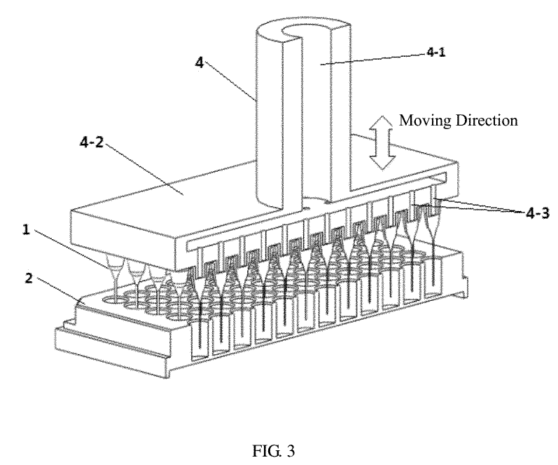

Referring to FIG. 3, one embodiment of the passage member 4 comprises one tubular inlet end 4-1, one rectangular communicating member 4-2, and a plurality of tubular outlet ends 4-3. The rectangular communicating member 4-2 defines a cavity therein communicating with the tubular inlet end 4-1 and the plurality of tubular outlet ends 4-3. The plurality of tubular outlet ends 4-3 are arranged in an array and spaced from each other at a distance corresponding to the distance between adjacent containers 3 in the container array 2. Each tubular outlet end 4-3 is configured to be connected to and in fluid communication with one micro-pipe 1. The container array 2 can be a standard microplate with a number of wells such as 24, 96, 384, or 1536 wells.

The fluid driving unit can further comprise a liquid storage member such as a reservoir or a tank. The liquid storage member can be hermetically connected to and in fluid communication with the fluid driving device. The liquid storage member is configured to continuously supply the first liquid to the fluid driving device. Referring to FIG. 1, the barrel of the syringe is the liquid storage member. The fluid driving unit can comprise more than one liquid storage members respectively accommodating different first liquids. The amount of liquid storage members can correspond to the amount of the fluid passages in the passage member.

In another embodiment, the fluid driving unit does not comprise a liquid storage member, nor does it contain any liquid, or only loads an inert liquid rather than a first liquid. The first liquid is only loaded in the micro-pipe, for example, aspirated into the micro-pipe from the outlet end. The upper end or the inlet end of the micro-pipe can be enlarged to form a liquid storage member to accommodate a sufficient amount of the first liquid. The fluid driving unit indirectly drives the first liquid by generating air pressure or by driving the inert liquid. One advantage of this embodiment is that the fluid driving unit does not touch the first liquid, which is a sample solution, and thus does not cause contamination to the sample solution. Thereby, the used fluid driving unit does not need to be replaced or cleaned, and only the used micro-pipe is to be replaced to lower the cost.

A size of the liquid storage member is not limited, and as the droplets to be generated are in micro-quantity, the size of the liquid storage member does not need to be very large and can be in a microliter level in some embodiments, such as 5 microliters to 500 microliters, and 5, 10, 20, 50, 100, 150, 200, 250, 300, 400, or 500 microliters.

### Mechanical Moving Unit

The mechanical moving unit is configured to move the outlet end of the micro-pipe into and out from the second liquid, thereby dispensing the micro-quantity of the first liquid into the second liquid. The mechanical moving unit can further be configured to move the container relative to the micro-pipe in plane thereby dispensing the first liquid to the container one by one or group by group.

In one embodiment, such as in FIG. 1, the mechanical moving unit can comprise the manipulator 6 to move the micro-pipe 1. The manipulator 6 can hold the passage member 4 thereby moving the micro-pipe 1 with the passage member 4. In one embodiment, the manipulator 6 is a three-dimensional locating device that is capable of moving in three directions along x, y, and z axes. The manipulator 6 moves the outlet end of the micro-pipe 1 vertically into and out of the second liquid in the container 3 and horizontally locate the micro-pipe 1 to different containers 3 thereby dispensing the droplets to the containers 3 one by one. In another embodiment, the manipulator 6 only moves the micro-pipe 1 vertically along the z-axis, and the mechanical moving unit further comprises the moving support 7 to carry the container 3 and move the container 3 therewith in a horizontal direction, such as along x-axis and y-axis.

In another embodiment, such as in FIG. 2, the moving support can be a vertical lifting support driven by a stepper motor. The vertical lifting support carries the container 3 to move therewith in a vertical direction along the z-axis thereby allowing the outlet end of the micro-pipe 1 in and out from the second liquid in the container 3. In the embodiment that the passage member 4 comprises a plurality of fluid passages connecting to a plurality of micro-pipes 1 one to one corresponding to the arrangement of the containers 3 of the container array 2, the vertical lifting support can complete the dispensing of the droplets to every container 3 of the container array 2 by only moving up and down vertically.

The mechanical moving unit can have a stable relative motion between the micro-pipe 1 and the container 3, such as in a uniform speed, to improve the dispensing accuracy.

### Control and Feedback Unit

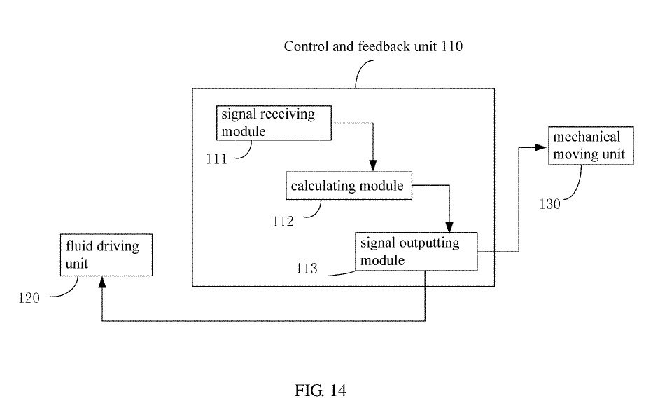

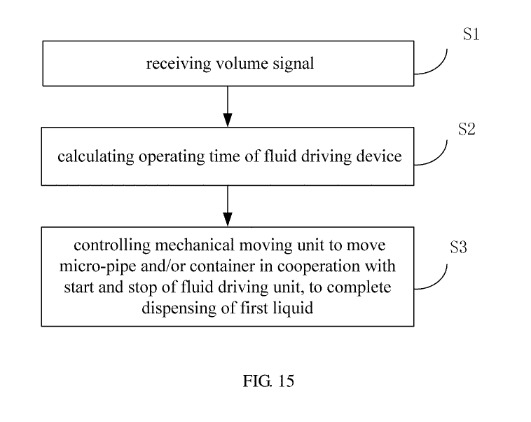

In one embodiment, the micro-quantity liquid dispensing/mixing apparatus can further comprise a control and feedback unit. The control and feedback unit can comprise a signal receiving module, a calculating module, and a signal outputting module. Referring to FIG. 14 and FIG. 15, the signal receiving module 111 of the control and feedback unit 110 receives a signal input by the user for deciding the volume of the first liquid to be dispensed, and transmits the signal to the calculating module 112 (S1). The calculating module 112 calculates a fluid driving time of the fluid driving device in the fluid driving unit 120 based on a pre-set value of a fluid driving speed, or calculates the fluid driving speed based on the volume of the first liquid to be dispensed. The calculating module 112 transmits the calculation result to the signal outputting module 113 (S2). The signal outputting module 113 controls the mechanical moving unit 130 to have a relative movement between the micro-pipe and the container in cooperation with the start and stop of the fluid driving unit 120, to complete the dispensing of the first liquid (S3).

The signal outputting module 113 controls the mechanical moving unit 130 to move the micro-pipe and/or the container, to locate the micro-pipe above the target container and to insert the outlet end of the micro-pipe below the liquid surface of the second liquid in the target container. Then the signal outputting module 113 controls the fluid driving unit 120 to drive the second liquid in the fluid driving speed for the fluid driving time. After the fluid driving unit 120 stops the driving of the first liquid, the signal outputting module 113 controls the mechanical moving unit 130 to have the outlet end of the micro-pipe drawn out from the second liquid through the liquid surface to complete the dispensing of the predetermined volume of the first liquid.

In one embodiment, the control and feedback unit further comprises a distance detecting module (not shown) to detect the relative positional relationship between the outlet end of the micro-pipe and the target container, and also to detect the distance between the outlet end of the micro-pipe and the liquid surface of the second liquid in the target container. The distance signal obtained by the distance detecting module is transmitted to the signal receiving module 111. The signal receiving module 111 receives the distance signal and transmits the distance signal to the calculating module 112. The calculating module 112 receives the distance signal, compares the distance signal with a pre-set distance value between the outlet end of the micro-pipe and the liquid surface of the second liquid in the target container, and transmits the compared result to the signal outputting module 113. The signal outputting module 113 receives the compared result, converts the compared result into a corresponding instruction signal, and transmits the instruction signal to the mechanical moving unit to adjust the movement of the mechanical moving unit till the outlet end of the micro-pipe is located at a predetermined distance above the liquid surface of the second liquid. For example, when the real distance is larger than the pre-set distance, the mechanical moving unit moves the micro-pipe towards the liquid surface of the second liquid.

In the embodiment having the container array, the control and feedback unit is capable of detecting whether the amount (or the liquid depth) of the second liquid in the target container is equal to or greater than a minimum value. For example, the distance detecting module detects a distance between the liquid surface of the second liquid and the bottom of the target container. When the second liquid in the target container is less than the minimum value, the signal outputting module outputs an alarm signal and controls the mechanical moving unit to stop dispensing the first liquid to the target container. For example, when the control and feedback unit detects that the target container has no second liquid, the alarm signal is output and the dispensing of the first liquid is stopped.

The liquid surface of the second liquid can be detected by an infrared laser ranging sensor or an ultrasonic ranging sensor.

In the embodiment having a plurality of containers, such as the 96-well microplate, the control and feedback unit 110 repeats the dispensing until the predetermined volume of the first liquid is dispensed into every containers. In the embodiment that the passage member has a plurality of fluid passages connecting the plurality of containers, the first liquid can be dispensed to all the containers at the same time.

The control and feedback unit 110 can simultaneously control the operation of a plurality of fluid driving units 120 and/or a plurality of mechanical moving units 130 respectively, in order to simultaneously dispense different volumes of liquid.

### Sterilization/Dedusting/Destaticizing and Gas Replacing Unit

In one embodiment, the apparatus further comprises a sterilization/dedusting/destaticizing unit to remove the microorganism and/or the dust and/or the electrostatic charge in the air around the apparatus. The sterilization/dedusting/destaticizing unit can form a closed space enclosing at least the micro-pipe and the container or the entire system, and can filter the air by an air filtering device in the closed space.

The sterilization/dedusting/destaticizing unit can further comprise a UV sterilization lamp mounted near the micro-pipe and the container.

The apparatus can further comprise a gas replacing unit to replace the gas in the apparatus, thereby having an inert gas protection, and anaerobic operation, and providing gas atmosphere required for cell culture or biochemical reaction such as carbon dioxide, oxygen gas, and hydrogen gas.

### Temperature Control and Feedback Unit

In one embodiment, the micro-quantity liquid dispensing/mixing apparatus can further comprise a temperature control and feedback unit for providing a desired temperature for the liquid system in the container. The temperature control and feedback unit can be arranged below a support having the container located thereon. The temperature control and feedback unit can comprise a heating device and/or a cooling device, and a temperature controlling device. The temperature control and feedback unit can directly heat and/or cool the support. In the embodiment where the apparatus comprises the moving support, the temperature control and feedback unit can directly heat and/or cool the moving support. In another embodiment, the temperature control and feedback unit can be an incubator to accommodate the container.

According to a second aspect of the present disclosure, a micro-quantity liquid dispensing system is provided. The micro-quantity liquid dispensing system comprises the above-described micro-quantity liquid dispensing/mixing apparatus, the above-described micro-pipe, and the above-described container.

### Micro-Pipe

The micro-quantity liquid dispensing system can comprise the micro-pipe. The micro-pipe is capable of outputting micro-quantity of liquid.

Referring to FIG. 2, the micro-pipe 1 is the capillary tube having two opposite ends defining openings respectively. One end of the capillary tube is the inlet end to connect the passage member 4. The other end of the capillary tube is the outlet end to dispense the first liquid. The first liquid in the capillary tube flows in the direction from the inlet end to the outlet end in dispensing. The material of the capillary tube can be metal (e.g., stainless steel), quartz, glass, or polymer.

The micro-pipe can be any tubular shaped structure that is capable of outputting a micro-quantity of liquid, such as a single-core capillary tube, a multi-core (or multi-cell) capillary tube, a capillary tube bundle, a capillary tube array, a sleeve tube enclosed capillary tube, or a microfluidic chip.

Referring to FIG. 3, in one embodiment, the micro-pipe 1 has an enlarged upper end as a reservoir for the first liquid.

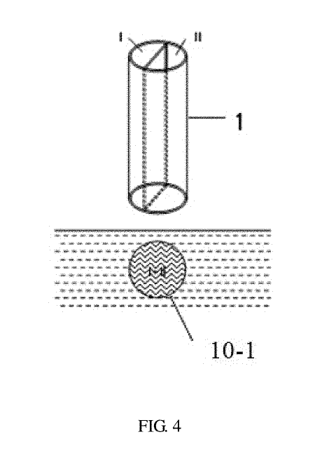

Referring to FIG. 4, one embodiment of the micro-pipe 1 is a double-core capillary tube comprising two separate liquid pathways, which are the first liquid pathway I and the second liquid pathway II. The double-core capillary tube comprises a tube and a separator inserted in the tube along the length direction and separating the inner space of the tube into the two liquid pathways I, II that are not in communication with each other. Two different first liquids can be filled in the two liquid pathways I, II respectively. Thereby, a mixed droplet 10-1 formed from the two first liquids can be generated. The two liquid pathways I, II can be in fluid communication with two fluid driving devices thereby the two first liquids in the two liquid pathways I, II can be driven in different speeds, to vary the ratio of the two first liquids in the droplet 10-1. In one embodiment, another two thinner capillary tubes are respectively inserted into the two semicircle shaped openings of the double-core capillary tube for loading the two first liquids. The double-core capillary tube can have an outer diameter of about 300 microns, an inner diameter of about 200 microns, and a thickness of the separator can be about 50 microns. In other embodiments, the multi-core capillary tube can have a plurality of cores such as three cores, four cores, or five cores.

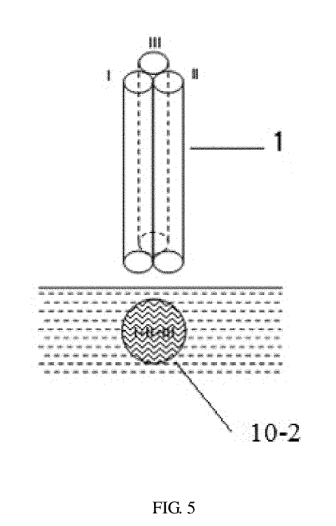

Referring to FIG. 5, one embodiment of the micro-pipe 1 is a capillary tube bundle comprises three separate capillary tubes I, II, III aligned along the same direction and stacked together. The three capillary tubes I, II, III can be respectively connected to three fluid driving devices to fill different first liquids in the three capillary tubes I, II, III, for example in the situation that the three first liquids that cannot be previously mixed. By using the capillary tube bundle 1, the mixed droplet 10-2 having the three first liquids can be generated. The capillary tube bundle 1 can comprise a plurality of capillary tubes, such as two, three, four, five, or even more capillary tubes.

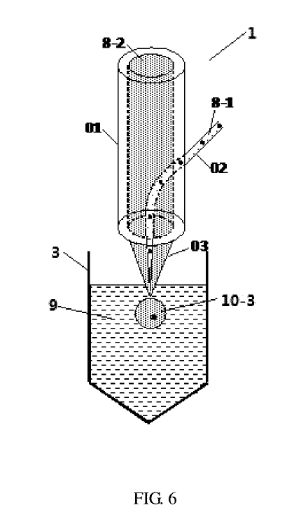

Referring to FIG. 6, one embodiment of the micro-pipe 1 is a sleeve tube enclosed capillary tube comprising a flexible tube 01, and a capillary tube 02 inserted into the flexible tube 01 from a side surface. The outlet end of the flexible tube 01 is a tapered end 03. The outlet end of the capillary tube 02 is located in the tapered end 03 of the capillary tube 02. The capillary tube 02 is filled with a first solution 8-1, and the flexible tube 01 is filled with a second solution 8-2. A mixed droplet 10-3 having the first solution 8-1 and the second solution 8-2 can be generated outside the tapered end 03. This sleeve tube enclosed capillary tube can simultaneously dispense a bulky liquid and a micro-quantity of the first liquid in the same system. For example, the sleeve tube enclosed capillary tube can be used to form a droplet comprising a cell suspension and a cell lysis buffer. A material of the flexible tube 01 can be such as rubber, latex, or silicone. In one embodiment, the flexible tube 01 is a rubber hose having a length of about 2 centimeters and an inner diameter of about 0.6 millimeters, the tapered end 03 is a Teflon® tube having an inner diameter of about 300 microns and an outer diameter of about 600 microns, the capillary tube 02 can be a glass capillary tube having an inner diameter of about 50 microns, an outer diameter of about 100 microns, and a length of about 10 centimeters, and the outlet end of the capillary tube 02 is pointed.

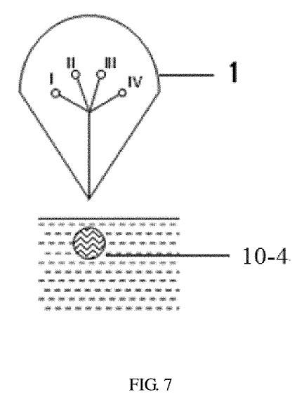

Referring to FIG. 7, one embodiment of the micro-pipe 1 is a microfluidic chip. The microfluidic chip has a tapered outlet end defining an outlet opening at the lower side and an inlet end at the upper side defining four inlet openings. The microfluidic chip defines four microchannels, which are a first microchannel I, a second microchannel II, a third microchannel III, and a fourth microchannel IV, and also defines a joining microchannel. The four microchannels are joined together and in fluid communication with the joining microchannel centered in its tapered probe. Each of the four microchannels is in fluid communication with an inlet opening. The joining microchannel is in fluid communication with the outlet opening. Thereby, the first liquids are introduced from the four openings of the four microchannels, joined together in the joining microchannel, and driven out from the opening of the joining microchannel at the pointed shaped outlet end. A droplet 10-4 can be formed outside the pointed shaped outlet end, which can then be dispensed in the second liquid in the container. The four microchannels can be in communication with four fluid driving devices loading with four different first liquids to form the droplet 10-4 comprising the four different first liquids mixed together.

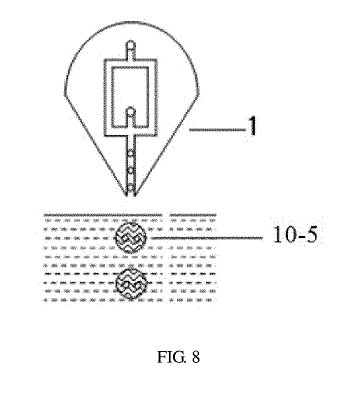

Referring to FIG. 8, one embodiment of the micro-pipe 1 is another microfluidic chip. The microfluidic chip has a pointed shaped outlet end at the lower side of the microfluidic chip and an inlet end at the upper side of the microfluidic chip. The pointed shaped outlet end defines an outlet opening. The inlet end defines a first inlet opening and a second inlet opening. The microfluidic chip defines two first microchannels and one second microchannel. The two first microchannels are both in fluid communication with the first inlet opening. The second microchannel is in fluid communication between the second inlet opening and the outlet opening.

The two first microchannels are joined and in fluid communication with the second microchannel at the same position from two opposite sides of the second microchannel. Thereby, the first liquid in the first microchannels can “cut” the third liquid (e.g., a reaction liquid) in the second microchannel and form a core-shell droplet 10-5. By varying the flow speeds of the first liquid and the third liquid, a ratio of the first liquid to the third liquid in the droplet 10-5 can be changed.

For example, the first liquid in the first microchannels can be oil, and the third liquid in the second microchannel can be water containing a sample. The droplet 10-5 can be a water-in-oil droplet. The second liquid in the container can be water. Thus, the droplet 10-5 dispensed in the second liquid can form a water-in-oil-in-water system.

It can be understood that FIG. 7 and FIG. 8 only show two specific examples of the microfluidic chip. Other microfluidic chips having different patterns of microchannels can be used as the micro-pipes if only the droplets can be formed outside the outlet ends of the micro-pipes 1.

The shape of the outlet end of the micro-pipe is configured to have the micro-quantity of the first liquid driven out from the micro-pipe formed into a droplet attached to the outlet end of the micro-pipe. An outer diameter of the outlet end of the micro-pipe can be in a range from about 0.05 microns to about 1000 microns, and in some embodiments can be about 5 microns to about 400 microns. The outlet end of the micro-pipe can be cylindrical, tapered, conical, or pointed. An inner diameter of the flow path defined in the micro-pipe at the outlet end can be in a range from about 0.025 microns to about 500 microns, and in some embodiments can be about 2.5 microns to about 200 microns, and in some embodiments can be about 10 microns to about 150 microns.

In one embodiment, the micro-pipe has a single flow path and a conical outlet end with an inner diameter of about 10 microns to about 100 microns and an outer diameter of about 20 microns to about 200 microns.

In another embodiment, the micro-pipe is a glass or quartz capillary tube having a conical outlet end with an inner diameter of about 30 microns and an outer diameter of about 50 microns.

The specific structure and size of the micro-pipe 1 can be selected depending on the needs, and is not particularly limited in the present disclosure.

In order to precisely control the amount of first liquid to be dispensed into and to make the generated droplets more uniform, the outer surface of the outlet end of the micro-pipe can be further treated to have a low surface energy so that the first liquid can be more easily detached from the outlet end. The outer surface of the outlet end of the micro-pipe can be coated with a low surface energy coating or can be silanized. In one embodiment, the outer surface of the outlet end of the micro-pipe is silanized by a perfluorosilane (e.g., 1H, 1H, 2H, or 2H-perfluorooctyltrichlorosilane, Fluorochem Ltd., Derbyshire, United Kingdom).

### Container

The micro-quantity liquid dispensing system can comprise the container. The container can be any suitable container, in the form of a single container, a one-dimensional array of containers, or two-dimensional array of containers. A commercially available microplate can be used as the container, such as the standard microplate with a number of wells, such as 24, 96, 384, or 1536 wells. The standard microplate after liquid dispensing can be used directly for further standardized experiment and/or testing to reduce the cost. Corresponding to the microplate, a plurality of micro-pipes can be arranged in a one-dimensional array or a two-dimensional array, which improve the throughput for dispensing of the first liquid.

The container is used to contain the second liquid and receive the first liquid. The container can also be used to store and transfer the droplet of the first liquid. For example, the container can also be used to dilute the droplet of the first liquid, to receive two or more droplets of the first liquids, or to fuse the two or more droplets into one larger droplet.

A volume of the container can be in a microliter lever or in a milliliter lever. A shape of the container is not limited, such that a well can be used as a container. For example, the container can be a polymerase chain reaction (PCR) microplate, a PCR tube, a test tube, a flat bottom container, or a conical bottom container.

The size and amount of containers are not particularly limited and can be selected by those skilled in the art depending on the particular needs.

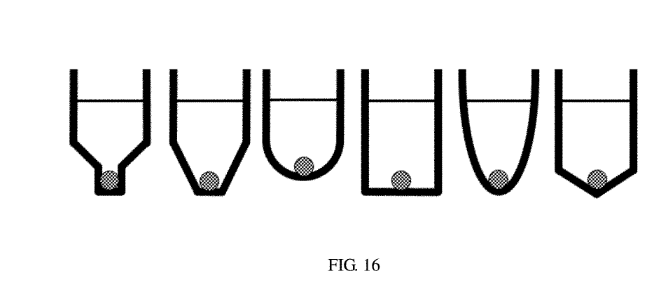

Referring to FIG. 16, the container can have a pointed bottom, a tapered bottom, a rounded bottom, a flat bottom, an oval bottom, or a conical bottom (as shown from left to right in FIG. 16).

### First Liquid

The type of the first liquid is not limited and is selected and prepared according to the needs. For example, the first liquid can be a solution containing a sample, a solution containing a reactant, or a solution or solvent for dilution, buffering, or solubilization. The first liquid can be a mixed solution of a plurality of solutions (such as in the embodiments of FIGS. 3 to 6), and can be an emulsion (such as in the embodiment of FIG. 8).

### Second Liquid

The second liquid is immiscible with the first liquid. The second liquid is usually inert to the first liquid. The liquid-liquid interface or the gas-liquid interface (e.g., the liquid surface) of the second liquid provides a shearing force for detaching the droplet from the micro-pipe.

The type of the second liquid is not particularly limited, and can be chemically stable and cannot react with the first liquid. The second liquid can be non-volatile and not interfere with a subsequent detection/reaction after the first liquid is dispensed into the second liquid. When the first liquid is an aqueous solution, the second liquid can be an oil phase. For example, the second liquid can be mineral oils (including n-tetradecane, etc.), vegetable oils, silicone oils, or perfluoroalkane oils. When the first liquid is an oil phase, the second liquid can be water or an aqueous solution such as deionized water, sterile water, or the like. The second liquid can also be an ionic liquid or a magnetic liquid.

The second liquid can provide an environment for the droplet of the first liquid to be stable presence, and can also provide a space for the droplet to be isolated from the external environment, avoiding the risk of contamination of the droplet, and avoiding the volatilization of first liquid that leads imprecise experimental results.

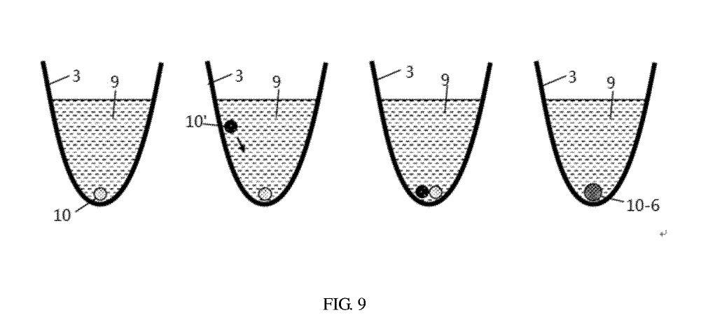

In one embodiment, the specific gravity of the first liquid is larger than that of the second liquid, so that the generated droplet sink to the bottom of the container, the droplet position can be easily detected, and multiple droplets can be easily aggregated and fused (such as in the embodiment of FIG. 9).

### Other Liquid And Solid

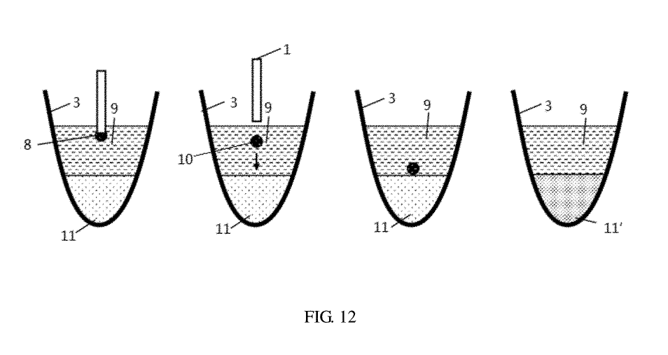

The apparatus, system, and method that are capable of dispensing micro-quantity of liquid can also comprise other liquid besides the first and second liquids, such as the third liquid. Referring to FIG. 12, in one embodiment, the container 3 contains the second liquid 9 and the third liquid 11 below the second liquid 9.

The droplet 10 of the first liquid 8 is dispensed into the second liquid 9 by drawing the outlet end of the micro-pipe 1 out from the second liquid 9. The droplet 10 descends under the action of gravity to the interface of the second liquid 9 and the third liquid 11. The third liquid 11 is miscible with the first liquid 8, and the droplet 10 of the first liquid is fused with the third liquid 11 under the action of interfacial tension to bring the substance in the first liquid 8 into the third liquid to form a solution 11.

In another embodiment, the third liquid is located above the second liquid. The first, second, and third liquids are immiscible with each other. The outlet end of the micro-pipe is inserted below the liquid level of the second liquid, the fluid driving device is started for a period of time to produce the required volume of the droplet of the first liquid, and then the micro-pipe is lifted to the third liquid. When the outlet end of the micro-pipe passes through the interface of the second and third liquids, the droplet is detached from the outlet end and left in the second liquid due to the surface tension of the interface. This embodiment can be used in situations where the second liquid is volatile or needed to be isolated from the external environment, such as in anaerobic or other situations. For example, the first liquid can be a sample solution or a reactant solution (aqueous solution having a specific gravity of about 1), the second liquid can be a silicone oil (with a specific gravity of about 0.9), and the third liquid can be a mineral oil (with a specific gravity of about 0.76).

The container can also contain a micro-bead, which is a liquid or a solid. The micro-bead comprises a reactant for reacting with the first liquid. The reactant can be dissolved in the liquid micro-bead or coated on the solid micro-bead. The droplet of the first liquid that is dispensed in the container is in physical or mechanical contact with the micro-bead. When the micro-bead is a liquid, it can be fused with the droplet of the first liquid to have the reaction. When the micro-bead is a solid particle, the droplet of the first liquid can be coated on the surface of the micro-bead thereby reacting with the reactant coated on the surface of the micro-bead.

### Method for Dispensing Micro-Quantity of Liquid

According to the principle of the present disclosure, one embodiment of the method for dispensing micro-quantity of liquid comprises steps of:

A, inserting the outlet end of the micro-pipe into the second liquid contained in the container;

B, driving the first liquid in the micro-pipe to move towards the outlet end, and forming a droplet of the first liquid having a predetermined volume outside the outlet end of the micro-pipe; and

C, drawing the outlet end of the micro-pipe out from the second liquid to detach the droplet having the predetermined volume from the outlet end of the micro-pipe thereby being dispensed in the second liquid;

wherein the first liquid is not miscible to the second liquid.

The dispensing volume accuracy of the present method is affected by the accuracy of the fluid driving device and the size of the outlet end of the micro-pipe, so that the method is capable of obtaining a relatively high accuracy of the micro-quantity of the first liquid.

For example, a commercially available micro-syringe pump, such as a PHD Ultra injection pump (Harvard Apparatus, USA) as the fluid driving device can have a minimum flow rate of 25 fL/s and an accuracy of ±0.25%. Correspondingly, the outlet end of a commercially available micro-pipe can have an inner diameter of 2 microns. Thus, in this embodiment, the theoretically minimal micro-quantity of liquid can be 50 fL and the absolute error can be less than ±5 fL.

The step B can comprise a step of forming the predetermined volume of the droplet by adjusting the flow speed and time of the first liquid in the micro-pipe. The predetermined volume of the droplet (or the predetermined volume of the micro-quantity of the first liquid to be dispensed into) can be adjusted in femtoliters to nanoliters, such as in a range from about 10 fL to about 10 μL, and can be in a range from about 2 pL to about 200 nL in some embodiments.

Referring back to FIG. 1 and FIG. 2, in one embodiment, the flow speed of the first liquid in the micro-pipe 1 can be adjusted by controlling the fluid driving speed of the fluid driving device 5. The flow speed can be, as an example, 4 nL/s, and the predetermined volume of the droplet can be 20 nL. At the beginning, the outlet end of the micro-pipe 1 is located above the liquid surface of the second liquid 9, and the first liquid is filled in the micro-pipe 1. The liquid surface of the first liquid 8 can be aligned with the opening of the outlet end of the micro-pipe 1. The micro-pipe 1 moves (e.g., vertically down) towards the second liquid 9. At a first moment, the outlet end of the micro-pipe 1 is inserted into the second liquid 9 and stopped. Then, the fluid driving device 5 starts to drive the first liquid 8 out from the outlet end of the micro-pipe 1 at a flow speed of about 4 nL/s. At a second moment when the driving time lasts for about 5 seconds, the driving of the first liquid 8 is stopped, and a droplet 10 with a volume of about 20 nL and a diameter of about 336.8 microns is formed outside and attached to the outlet end of the micro-pipe 1. Then, the micro-pipe 1 is moved (e.g., vertically up) to a position out of the second liquid 9 and away from the liquid surface of the second liquid 9. At the moment when the outlet end of the micro-pipe 1 passes through the liquid surface of the second liquid 9, the droplet 10 is detached from the outlet end of the micro-pipe 1 by action of the surface tension of the liquid surface. The droplet 10 is left in the second liquid 9 and sinks to the bottom of the container 3. As the container 3 has a rounded bottom, the droplet 10 can sink to the center of the bottom due to gravity. The micro-pipe 1 can then be moved to the next container 3 to complete another dispensing of the 20 nL of the first liquid 8 by repeating the above-described steps.

The micro-pipe 1 can be moved in a uniform speed up and down to avoid disturbing and affecting the accuracy of the droplet volume.

In another embodiment, the micro-pipe 1 can be static, and the container 3 can be moved vertically up and down.

When there is a need to mix two or more liquids, the liquids can be mixed before dispensing, and the first liquid having the mixed liquids can be dispensed at one time. Or, in another embodiment, referring to FIG. 9, the liquids can be mixed after being dispensed in the second liquid. Two or more droplets 10, 10′ of different first liquids can be dispensed in the second liquid 9 at separate times one by one. The two or more droplets 10, 10′ can be aggregated at the bottom of the container 3 and fused to form a mixed droplet 10-6 in the second liquid 9.

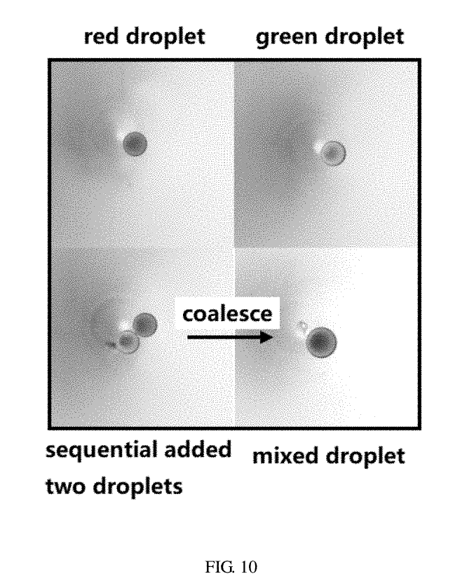

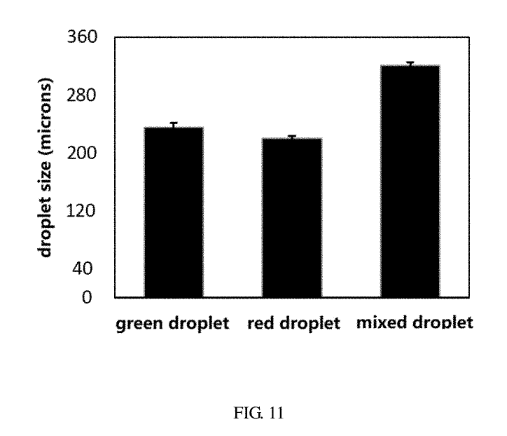

Referring to FIG. 10, which shows two droplets each having a different color fusing into one droplet. The upper left droplet is a red droplet and the upper right droplet is a green droplet, both of which are about 5 nL. The lower left and lower right figures show a fusion of the red and green droplets into one brown (red+green) droplet. The spontaneous fusion can be relatively slow only under the action of gravity, such as in 1 minute to 1 hour. In order to accelerate the fusion of the droplets, the container can be placed in a low-speed centrifuge and rotated at about 2000 rpm for about 30 seconds to achieve the droplet fusion. Referring to FIG. 11, the average diameters of the red, green and brown (red+green) droplets in 5 parallel experiments are shown, revealing that the diameters of the droplets have good reproducibility.

Referring to FIG. 12, in one embodiment, the micro-quantity of the first liquid is dispensed and diluted in a large amount of liquid. For example, 100 microliters of an aqueous liquid 11 is contained in the rounded bottom container 3 and covered with oil 9. 5 nL of droplet 10 is dispensed in the container by using the above-described steps. The droplet 10 sinks under gravity, and is dissolved in the aqueous liquid 11 and thus is diluted about 2000 times. The micro-pipe 1 is only in direct contact with the oil 9, and is not in contact with the aqueous liquid 11 thereby avoiding contamination and increasing the reliability of the method.

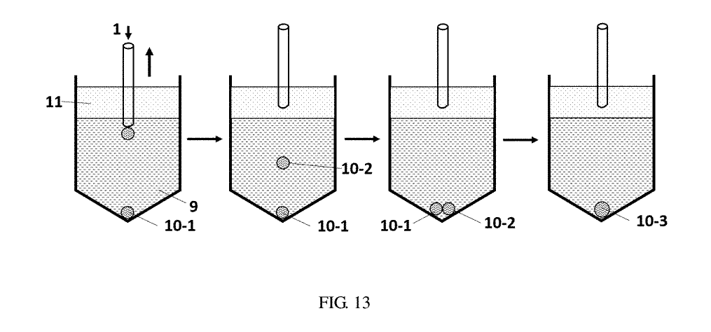

Referring to FIG. 13, besides the first liquid and the second liquid, the third liquid is used to protect the first liquid during the dispensing. For example, 100 microliters of the second liquid 9 which is a first oil is contained in the tapered bottom container 3, and the third liquid 11 which is a second oil and is not soluble with the first oil is covering above the first oil. The droplet 10-1 of one aqueous first liquid is dispensed at the tapered bottom of the container 3. The micro-pipe 1 is inserted into the second liquid 9 below the liquid surface of the third liquid 11 to dispense the droplet 10-2 of the first liquid by drawing the outlet end of the micro-pipe 1 from the second liquid 9. The droplet 10-1 and the droplet 10-2 can be fused in the second liquid 9 to form a bigger droplet 10-3. The third liquid 11 can have a gas insulating function. During the dispensing and fusing, the droplets 10-1 and 10-2 are always below the third liquid 11 thereby protected by the third liquid 11. So that the probability of contamination declines, and the method is conducive to improving the reliability of analysis.

Since the apparatus, system, and method of the present disclosure are simple, inexpensive, and easy to operate, a high-throughput liquid dispensing can be achieved, and the dispensing volume of the first liquid can be easily adjusted with a high accuracy. Therefore, the apparatus, system, and method of the present disclosure have a broad application prospect in the fields such as drug screening, cytotoxic study, protein crystallizing condition screening, single cell enzyme activity analysis, single cell whole genome sequencing and transcriptome sequencing, digital PCR quantitative nucleic acid amplification analysis, and cell-cell interaction study.

Method for Fusing Micro-Droplets

In the present disclosure, one embodiment of a method for fusing micro-droplets is also provided, and the method comprises:

S11, providing an oil based second liquid comprising an oil-soluble surfactant in a container; and

S12, dispensing two or more micro-droplets of a water based first liquid in the oil based second liquid.

The oil based second liquid comprises oil and the oil-soluble surfactant mixed therewith. The oil-soluble surfactant is a surfactant that is capable of being dissolved in oil. The oil-soluble surfactant can be at least one of Span® 20, Span® 40, Span® 60, Span® 80, Tween® 85, polyglycerol fatty ester, tertiary alkyl amines, stearic acid triethanolamine quaternaries (EQDMS), tri-(stearyl hydroxyethyl) methylammonium methylsulfates, cetyl polyethylene glycol/polypropylene glycol-10/1 dimethylsiloxane (ABIL® EM 90 or ABIL® EM 180), and oleic diethanolamide (ODEA).

A volume percentage of the oil-soluble surfactant in the oil based second liquid can be in a range from about 0.01% to about 0.5%.

In one embodiment, the oil-soluble surfactant is ABIL®EM 90 or ABIL®EM 180, and the volume percentage of ABIL®EM 90 or ABIL®EM 180 in the oil based second liquid is about 0.06% to about 0.5%. In another embodiment, the oil-soluble surfactant is Span® 80, and the volume percentage of Span® 80 in the oil based second liquid is about 0.01% to about 0.05%.

The oil in the oil based second liquid can be at least one of vegetable oil, mineral oil, and synthetic oil. The vegetable oil can comprise at least one of peanut oil, rapeseed oil, sunflower oil, castor oil, and tea seed oil. The mineral oil can comprise at least one of low boiling mineral oil and light mineral oil. The synthetic oil can comprise at least one of light silicone oil, dimethyl silicone oil, squalane, isohexadecane, isooctyl palmitate, isopropyl palmitate, caprylic/capric triglyceride, ethyl oleate, tridecyl stearate, and isooctyl stearate.

The water based first liquid that the droplets made of comprises water. In one embodiment, the water based first liquid further comprises a water-soluble surfactant. The water-soluble surfactant can be at least one of nonionic surfactant, anionic surfactant, or cationic surfactant. The nonionic surfactant can comprise at least one of polyethylene glycol having a molecular weight between 100 and 20,000, polypropylene glycol having a molecular weight between 100 and 2500, sorbitol, glycerol, sucrose ester, alkyl polyglucoside, octylphenylpolyethylene glycol, Tween® 80, Tween® 60, and dimethyl sulfoxide. The anionic surfactant can comprise at least one of sodium dodecylsulfonate (SDS), triethanolamine, and dioctyl sulfosuccinates. The cationic surfactant can comprise at least one of diethanolamides, triethanolamines, hexadecyl trimethyl ammonium bromide, cetyltrimethylammonium bromide (CTAB), and alkyl dimethyl ammonium chloride (ADAC).

The method for fusing micro-droplets can further comprise: S14, fusing the two or more micro-droplets of the water based first liquid in the oil based second liquid by a force that is capable of fuse the two or more micro-droplets.

In S14, under the action of the force, the two or more micro-droplets can be in mechanical contact to achieve a fusion with each other. The force can be such as a centrifugal force, a vibration force, an electromagnetic force, and combinations thereof.

The method for fusing micro-droplets can further comprise:

S13, sinking the two or more micro-droplets to the bottom of the container before S14.

In S13, by using the container with a narrower bottom, such as the pointed bottom, round bottom, oval bottom, or tapered bottom, and by having the specific gravity of the first liquid larger than that of the second liquid, the two or more micro-droplets can be sunk to the center of the bottom and aggregated together.

The method for fusing micro-droplets can further comprise:

S10, eliminating static electricity of the environment before S12.

In S10, the elimination of static electricity in the environment can comprise grounding the instrument, electrostatic shielding, and/or increasing environment humidity.

### Micro-Quantity Liquid Aspirating and Dispensing Apparatus

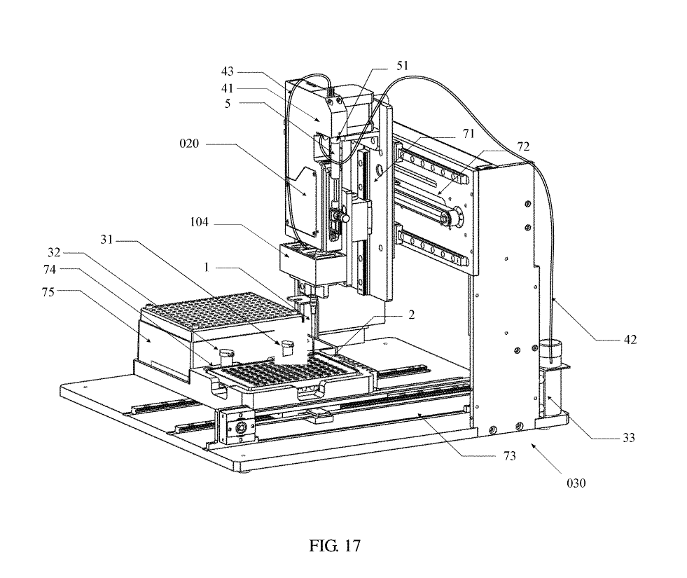

In the present disclosure, another embodiment of a micro-quantity liquid dispensing/mixing apparatus is also provided, which is a micro-quantity liquid aspirating and dispensing apparatus. Referring to FIG. 17, the apparatus comprises a micro-pipe 1, a fluid driving unit 020, and a mechanical moving unit 030.

The fluid driving unit 020 comprises a fluid driving device 5, a three-way valve 41, an oil conduit 42, and an outlet conduit 43. The three-way valve 41 comprises a first port connected to and in fluid communication with the fluid driving device 5, a second port connected to and in fluid communication with the oil conduit 42, and a third port connected to and in fluid communication with the outlet conduit 43. The three-way valve 41 is capable of selectively fluid communicating two of the fluid driving device 5, the oil conduit 42, and the outlet conduit 43, while fluid insulating the third.

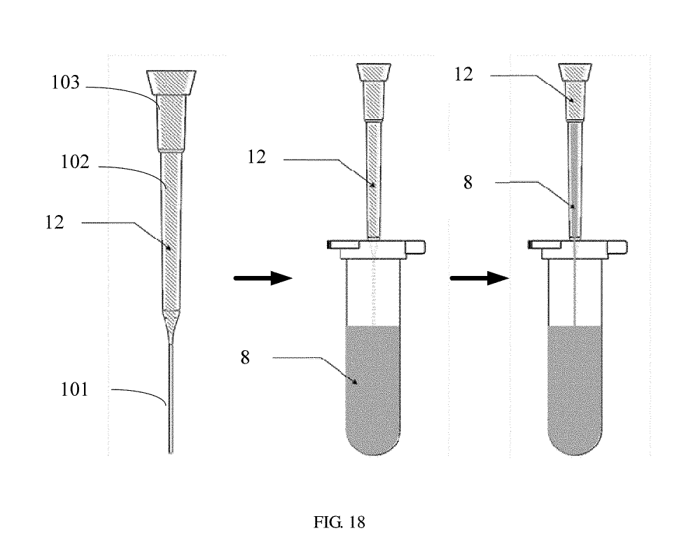

Referring to FIG. 18, the micro-pipe 1 can comprise an outlet end 101, a liquid storage section 102, and an inlet end 103 from bottom to top. Besides the dispensing of the first liquid, the micro-pipe 1 is also configured to perform a suction of the first liquid 8 from the outlet end of the micro-pipe 1. The inlet end 103 of the micro-pipe 1 is connected to and in fluid communication with the outlet conduit 43. The apparatus can further comprise a micro-pipe connector 104 configured to mount the micro-pipe 1 and connect the micro-pipe 1 with the outlet conduit 43.

The outlet conduit 43 comprises two opposite ends respectively connected to the micro-pipe 1 and the three-way valve 41, thereby fluid communicating the three-way valve 41 with the micro-pipe 1.

The oil conduit 42 is in fluid communication with an oil storage reservoir 33 containing oil, which is used to insulate the first liquid from the air.

The fluid driving device 5 is capable of driving (e.g., pushing and pulling) the fluid therein. By fluid communicating the fluid driving device 5 with the outlet conduit 43 and the oil conduit 42 respectively, the fluid driving device 5 can drive the fluid (e.g., the oil) in the outlet conduit 43 and the oil conduit 42. By communicating the fluid driving device 5 with the oil conduit 42 through the three-way valve 41, and fluid insulating the outlet conduit 43 from the fluid driving device 5 and the oil conduit 42 by the through the three-way valve 41, the fluid driving device 5 is capable of aspirating the oil from the oil storage reservoir 33 to the fluid driving device 5. By communicating the fluid driving device 5 with the outlet conduit 43 through the three-way valve 41, and fluid insulting the oil conduit 42 from the fluid driving device 5 and the outlet conduit 43 by the through the three-way valve 41, the fluid driving device 5 is capable of filling the oil from the fluid driving device 5 to the outlet conduit 43 and the micro-pipe 1. When the entire outlet conduit 43 and the micro-pipe 1 are filled with the oil, the air can be eliminated therefrom.

The mechanical moving unit 030 comprises a z-axis (vertical direction) moving support 71, an x-axis (one horizontal direction) moving support 72, and a y-axis (another horizontal direction) moving support 73. The z-axis moving support 71 has one end connected to the x-axis moving support 72 and another end connected to the fluid driving unit 020. Thereby, the fluid driving unit 020 is capable of moving along the z-axis and the x-axis with the z-axis moving support 71 and the x-axis moving support 72 correspondingly.

The apparatus can further comprise a microplate holder 74 configured to fix the microplate 2 on the y-axis moving support 73. The wells of the microplate 2 contain the second liquid and receive the micro droplets of the first liquid. The y-axis moving support 73 is capable of moving along the y-axis thereby moving the microplate 2 along the y-axis therewith. The apparatus can further comprise a micro-pipe storage box 11 mounted on the y-axis moving support 73.

In use, a waste oil storage reservoir 31 can be provided to receive the excess oil output from the outlet end 101 of the micro-pipe 1. The mechanical moving unit 030 is configured to move the fluid driving unit 020 such that the outlet end 101 of the micro-pipe 1 is located above the waste oil storage reservoir 31, and the fluid driving unit 020 is configured to fill the oil fully in the outlet conduit 43 and the micro-pipe 1 to eliminate air therein.

A first liquid storage reservoir 31 containing the first liquid 8 can also be provided. After the air in the outlet conduit 43 and the micro-pipe 1 is eliminated by the oil, the mechanical moving unit 030 is configured to move the fluid driving unit 020 such that the outlet end 101 of the micro-pipe 1 is located above the first liquid storage reservoir 31 and the outlet end 101 of the micro-pipe 1 is then inserted into the first liquid 8 in the first liquid storage reservoir 31. The fluid driving device 5 is configured to aspirate the first liquid 8 from the first liquid storage reservoir 31 to the micro-pipe 1 through the outlet end 101. The amount of the first liquid 8 is controlled by the fluid driving device 5 to not overflow the liquid storage section 102 preventing direct contact to the micro-pipe connector 104 or the outlet conduit 43, which may induce a contamination to the first liquid 8.

When the first liquid 8 is loaded in the micro-pipe 1, the mechanical moving unit 030 is configured to move the fluid driving unit 020 such that the outlet end 101 of the micro-pipe 1 is located above a target well on the microplate 2 and inserting the outlet end 101 of the micro-pipe 1 into the second liquid in the target well to dispense the micro-droplet of the first liquid into the target well by using the above-described method.

The apparatus can further comprise a temperature detector 51 on the fluid driving device 5 to detect the temperature of the fluid driving device 5 to prevent an inaccuracy of the dispensing volume of the first liquid 8 induced by thermal expansion of the apparatus.

### Micro-Droplet Containing Microplate

Referring to FIG. 16, one embodiment of a micro-droplet containing microplate is also provided. The micro-droplet containing microplate comprises a microplate defining a plurality of wells thereon, a second liquid contained in the well, and at least one micro-droplet located in the second liquid. The well can be open to the environment or sealed from the environment. The micro-droplet is immiscible with the second liquid and has a greater specific gravity than the second liquid. The micro-droplet can be in solid state or liquid state. Each well can comprise at least one micro-droplet. The micro-droplets in one well can be solid micro-droplets, liquid micro-droplets, or a combination thereof. The micro-droplet can sink to the bottom of the well. The solid state micro-droplet can be formed by solidifying the micro-droplet of the first liquid dispensed by the above-described method and apparatus.

In one embodiment, the second liquid can be oil such as mineral oil, liquid alkane, ester, silicone oil, alkene, or semi-solid saturated hydrocarbon. The semi-solid saturated hydrocarbon can be such as paraffin or petroleum jelly (e.g., Vaseline®) and its mixture with liquid alkane or mineral oil.

### Micro-Quantity of Nucleic Acid Amplification Method

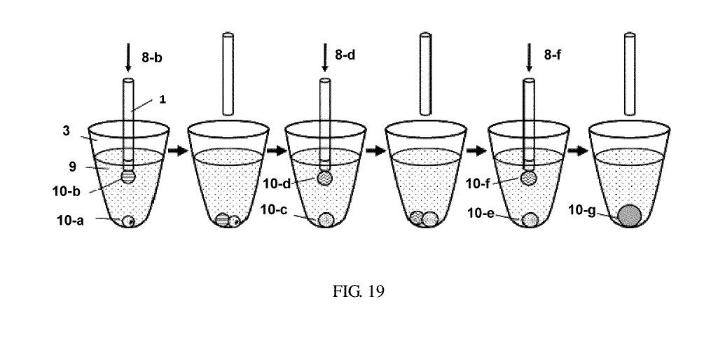

Referring to FIG. 19, in the present disclosure, one embodiment of a method for amplifying micro-quantity of nucleic acid of cell is also provided, and the method comprises: S21, providing a container 3 containing an oil phase 9;

S22, dispensing a micro-droplet of cell containing liquid 10-a in the oil phase 9 of the container 3;

S23, dispensing a micro-droplet of cell lysis buffer 10-b in the oil phase 9 of the container 3; S24, fusing the micro-droplet of cell containing liquid 10-a and the micro-droplet of cell lysis buffer 10-b into a first mixed droplet 10-c in the oil phase 9 of the container 3 to have a cell lysis and form nucleic acid in the first mixed droplet 10-c;

S25, dispensing a micro-droplet of stopping agent 10-d in the oil phase 9 of the container 3;

S26, fusing the micro-droplet of stopping agent 10-d and the first mixed droplet 10-c into a second mixed droplet 10-e to stop the cell lysis;

S27, dispensing a micro-droplet of amplification agent 10-f in the oil phase 9 of the container 3; and S28, fusing the micro-droplet of amplification agent 10-f and the second mixed droplet 10-e into a third mixed droplet 10-g to have a nucleic acid amplification.

The oil phase 9 is immiscible with any of the micro-droplets that are dispensed therein and has a smaller specific gravity than any of the micro-droplets that are dispensed therein. The container 3 can have a narrower bottom compared with the top in order to sink the micro-droplets to the center of the bottom of the container 3 thereby aggregating the micro-droplets together. The oil phase 9 can be such as mineral oil, liquid alkane, ester, silicone oil, and combinations thereof. In one embodiment, the oil phase 9 is mineral oil, which is biocompatible, resistant to high temperature, and has a low cost.

The micro-droplet of cell containing liquid 10-a can have a volume in femtoliter level to nanoliter level. The cell can be a live cell of animal, plant, or microorganism. The cell containing liquid can be a cell suspension. Each micro-droplet of cell containing liquid 10-a can comprise one cell, a plurality of cells, or a cell aggregation.

Single cell separation and extraction in generating the micro-droplet of cell containing liquid 10-a can be accomplished by a conventional method such as microdissection, micro-absorption, gradient dilution, flow cytometry, optical tweezering, or microfluidic sorting.

In S23, the micro-droplet of cell lysis buffer 10-b can be dispensed by inserting the outlet end of the micro-pipe 1 containing the cell lysis buffer into the oil phase 9; driving the cell lysis buffer out from the outlet end of the micro-pipe 1 to generate the micro-droplet of cell lysis buffer 10-b attached to the outlet end of the micro-pipe 1; and drawing the outlet end of the micro-pipe 1 out from the oil phase 9 thereby cutting the micro-droplet of cell lysis buffer 10-b from the outlet end of the micro-pipe 1 by the liquid surface of the oil phase 9, to detach the micro-droplet of cell lysis buffer 10-b from the outlet end of the micro-pipe 1 and being left in the oil phase 9.

The cell lysis buffer can be such as strong acid, strong alkali, surfactant, hypotonic solution, protease, lysozyme, combinations thereof, or liquid solution thereof. In one embodiment, the cell lysis buffer is an alkali solution.

In S24, the method for cell lysis can be decided based on types of the cell, and can be such as physical method, chemical method, or biological method. In one embodiment, the cell lysis is performed at a high temperature.

In S25, the micro-droplet of stopping agent 10-d can be dispensed by inserting the outlet end of the micro-pipe 1 containing the stopping agent into the oil phase 9; driving the stopping agent out from the outlet end of the micro-pipe 1 to generate the micro-droplet of stopping agent 10-d attached to the outlet end of the micro-pipe 1; and drawing the outlet end of the micro-pipe 1 out from the oil phase 9 thereby cutting the micro-droplet of stopping agent 10-d from the outlet end of the micro-pipe 1 by the liquid surface of the oil phase 9, to detach the micro-droplet of stopping agent 10-d from the outlet end of the micro-pipe 1 and being left in the oil phase 9.

The stopping agent is capable of stopping the cell lysis. In one embodiment, the stopping agent is an acid solution to neutralize the cell lysis buffer, which is the alkali solution.

In S27, the micro-droplet of amplification agent 10-f can be dispensed by inserting the outlet end of the micro-pipe 1 containing the amplification agent into the oil phase 9; driving the amplification agent out from the outlet end of the micro-pipe 1 to generate the micro-droplet of amplification agent 10-f attached to the outlet end of the micro-pipe 1; and drawing the outlet end of the micro-pipe 1 out from the oil phase 9 thereby cutting the micro-droplet of amplification agent 10-f from the outlet end of the micro-pipe 1 by the liquid surface of the oil phase 9, to detach the micro-droplet of amplification agent 10-f from the outlet end of the micro-pipe 1 and being left in the oil phase 9.

In one embodiment, two or more micro-droplets of different amplification agents can be respectively dispensed through different micro-pipes 1. The outlet end of the micro-pipe 1 can have an inner diameter of about 30 microns to 300 microns.

A volume of the micro-droplet of amplification agent 10-f can be about 50 nL to about 900 nL. The amplification agent can be a whole genome amplification solution such as an amplification solution used in multiple displacement amplification (MDA), degenerate oligonucleotide primed PCR (DOC-PCR), multiple annealing and looping-based amplification cycles (MALBAC), and so on. The amplification solution can also be a common PCR solution or other constant temperature amplification solutions, such as that used in loop-mediated isothermal amplification (LAMP), rolling circle DNA amplification (RCA), and so on.

In one specific embodiment, the outlet end of the micro-pipe 1 has an inner diameter of about 50 microns and an outer diameter of about 150 microns. The micro-droplet of cell containing liquid 10-a has a volume of about 2 nL. The micro-droplet of cell lysis buffer 10-b has a volume of about 30 nL. The container 3 having the oil phase 9 and the first mixed droplet 10-c is heated at about 65° C. for about 10 minutes to have the cell lysis and form the nucleic acid. The micro-droplet of stopping agent 10-d has a volume of about 30 nL. The micro-droplet of amplification agent 10-f has a volume of about 300 mL. The container 3 having the oil phase 9 and the third mixed droplet 10-g is incubated at about 37° C. for about 10 hours to have a nucleic acid amplification in a real-time fluorescence quantitative PCR instrument.

Finally, it is to be understood that the above-described embodiments are intended to illustrate rather than limit the present disclosure. Variations may be made to the embodiments without departing from the spirit of the present disclosure as claimed. Elements associated with any of the above embodiments are envisioned to be associated with any other embodiments. The above-described embodiments illustrate the scope of the present disclosure but do not restrict the scope of the present disclosure.

What is claimed is:

1. An apparatus for dispensing or mixing a micro-quantity of liquid, the apparatus comprising:
a fluid driving unit comprising a fluid driving device, the fluid driving device being configured to drive a micro-quantity of a first liquid in a micro-pipe out from an outlet end of the micro-pipe, the micro-quantity of the first liquid is attached to the outlet end of the micro-pipe; and
a mechanical moving unit configured to move the outlet end of the micro-pipe into a second liquid in a container and out from the second liquid;
wherein the fluid driving device and the mechanical moving unit are further configured to cooperate with each other to dispense the micro-quantity of the first liquid attached to the outlet end of the micro-pipe into the second liquid, and to detach the micro-quantity of the first liquid from the outlet end of the micro-pipe by drawing the outlet end of the micro-pipe out from the second liquid.

2. The apparatus of claim 1, wherein a volume of the micro-quantity of the first liquid is in a range from about 10 femtoliters to about 10 microliters.

3. The apparatus of claim 1, wherein the fluid driving unit further comprises a passage member defining at least one fluid passage, the passage member is joined with the fluid driving device, and the passage member is configured to have the micro-pipe joined thereto to couple and communicate the fluid driving device with the micro-pipe.

4. The apparatus of claim 3, wherein the passage member defines a plurality of fluid passages configured to communicate with a plurality of micro-pipes in a one-to-one manner.

5. The apparatus of claim 1, further comprising a control and feedback unit, wherein the control and feedback unit comprises:
a signal receiving module configured to receive a volume signal and transmit the volume signal to the calculating module, the volume signal representing a volume of the micro-quantity of the first liquid;
a calculating module configured to calculate a fluid flow speed and a fluid flow time based on the volume signal and output a calculation result comprising the fluid flow speed and the fluid flow time; and
a signal outputting module configured to receive the calculation result, form instruction signals based on the calculation result, and output the instruction signals respectively to the fluid driving unit and the mechanical moving unit to control cooperation between the fluid driving unit and the mechanical moving unit.

6. A system for dispensing or mixing micro-quantity of liquid, the system comprising:
a micro-pipe comprising an outlet end configured to contain a first liquid;
a container configured to contain a second liquid;
a fluid driving unit comprising a fluid driving device, the fluid driving device being configured to drive a micro-quantity of the first liquid in a micro-pipe out from an outlet end of the micro-pipe, the micro-quantity of the first liquid is attached to the outlet end of the micro-pipe; and
a mechanical moving unit configured to move the outlet end of the micro-pipe into the second liquid and draw out the outlet end of the micro-pipe from the second liquid;
wherein the fluid driving device and the mechanical moving unit are configured to cooperate with each other to dispense the micro-quantity of the first liquid attached to the outlet end of the micro-pipe into the second liquid, and to detach the micro-quantity of the first liquid from the outlet end of the micro-pipe by drawing the outlet end of the micro-pipe out from the second liquid.

7. The system of claim 6, wherein an outer diameter of the outlet end of the micro-pipe is in a range from about 0.05 microns to about 1000 microns, and an inner diameter of the outlet end is in a range from about 0.025 microns to about 500 microns.

8. The system of claim 6, wherein the micro-pipe is a single-core capillary tube, a multi-core capillary tube, a capillary tube bundle, a capillary tube array, a sleeve tube enclosed capillary tube, or a microfluidic chip.

9. The system of claim 6, wherein the container is a plurality of containers, the micro-pipe is a plurality of micro-pipes arranged according to the plurality of containers, the plurality of micro-pipes are insertable into the plurality of containers simultaneously.

10. The system of claim 6, wherein the container is a well of a microplate.

11. The system of claim 6, wherein the fluid driving unit further comprises an oil conduit, an outlet conduit, and a three-way valve;
the oil conduit is configured to be in communication with an oil storage reservoir;
the outlet conduit is configured to be in communication with the micro-pipe; and
the three-way valve is configured to communicate the fluid driving device with the oil conduit, and fluid insulating the outlet conduit from the fluid driving device and the oil conduit, thereby the fluid driving device being capable of aspirating the oil from the oil storage reservoir to the fluid driving device;
the three-way valve is further configured to communicate the fluid driving device with the outlet conduit, and fluid insulate the oil conduit from the fluid driving device and the outlet conduit, thereby the fluid driving device being capable of filling the oil from the fluid driving device to the outlet conduit and the micro-pipe; and
the fluid driving device is configured to aspirate the first liquid from a first liquid storage reservoir to the micro-pipe through the outlet end.

12. A method for dispensing or mixing micro-quantity of liquid, the method comprising:
inserting an outlet end of a micro-pipe into a second liquid contained in a container, a first liquid being in the micro-pipe;
driving a micro-quantity of the first liquid out from the outlet end of the micro-pipe and forming a droplet of the first liquid outside the outlet end of the micro-pipe; and
drawing the outlet end of the micro-pipe out from the second liquid to detach the droplet of the first liquid from the outlet end of the micro-pipe thereby dispensing the droplet of the first liquid in the second liquid;
wherein the first liquid is immiscible with the second liquid.

13. The method of claim 12, wherein the droplet of the first liquid is detached from the outlet end of the micro-pipe under an action of the liquid surface of the second liquid.

14. The method of claim 12, wherein a volume of the droplet of the first liquid is in a range from about 10 femtoliters to about 10 microliters.

15. The method of claim 12, wherein a specific gravity of the first liquid is greater than a specific gravity of the second liquid.

16. The method of claim 12 further comprising controlling a volume of the droplet of the first liquid by controlling a flow speed and a flow time of the first liquid in the micro-pipe.

17. The method of claim 12, further comprising:
dispensing two or more droplets of the first liquid in the container; and
fusing the two or more droplets of the first liquid into one bigger droplet.

18. The method of claim 12, further comprising:
filling the micro-pipe with a sealing liquid; and
aspirating the first liquid into the micro-pipe filled with the sealing liquid from the outlet end of the micro-pipe, thereby insulating the first liquid by the sealing liquid from a side away from the outlet end of the micro-pipe.

19. The method of claim 12, wherein the second liquid comprises oil and an oil-soluble surfactant.

20. The method of claim 12, wherein the container further contains a third liquid or a micro-bead; the third liquid is a liquid cover above the second liquid or a diluent for diluting the first liquid; the micro-bead comprises a reactant for reacting with the first liquid, and the micro-bead is a liquid micro-bead or a solid micro-bead.
